(19) United States
(12) Patent Application Publication
(10) Pub. No.: US 2006/0263298 A1
(43) Pub. Date: Nov. 23, 2006

(54) Assay

(76) Inventors: Li-ming Gan, Molndal (SE); Lars-Eric Marberg, Molndal (SE)

Correspondence Address: ASTRAZENECA R&D BOSTON, 35 GATEHOUSE DRIVE, WALTHAM, MA, 02451-1215, US

(21) Appl. No.: 10/555,586

(22) PCT Filed: May 5, 2004
(86) PCT No.: PCT/GB04/01954

§ 371 Date: Nov. 4, 2005

(30) Foreign Application Priority Data
May 10, 2003 (GB) 0310818.0

(51) Int. Cl. A61K 49/00

(52) U.S. Cl. 424/9.2

(57) ABSTRACT

The present invention relates to an ex vivo plaque rupture model and methods of use thereof, in particular as a screen for atherosclerosis plaque stabilizing drugs. The invention also relates to an apparatus used in the model and methods of use thereof.

The present invention concerns a novel ex vivo plaque rupture model, its uses, in particular as a screen for atherosclerosis plaque stabilising drugs, and apparatus used in the model.

Atherosclerosis is an arterial disease with typical progressive steps:

1. endothelial dysfunction and fatty-streak formation;

2. advanced fibrous lesion formation;

3. plaque rupture followed by thrombosis.

A plaque is an atherosclerotic lesion in the intima of an arterial wall with morphology characterized by a lipid core covered by a fibrotic cap.

Rupture of an atherosclerotic plaque followed by thromboembolic events is considered the major pathophysiological mechanism leading to acute myocardial infarction and stroke. Morphological data based on autopsy material indicates that a vulnerable plaque is not necessarily large but is often characterized by a thin fibromuscular cap and a large lipid core with high content of macrophages. These compositional characteristics of the vulnerable plaque have also been confirmed in series of animal experimental studies. Recently, by using MRI technique, Yuan et al., (Circulation. 105(2):181-5, 2002) were able to measure cap thickness of human carotid plaques and found that a thin cap is a strong predictor for cerebrovascular events such as stroke and transient ischemic attack (TIA).

In vivo, the vascular wall is continuously exposed to a complex array of biomechanical forces, such as shear stress (blood flow), intraluminal pressure (blood pressure) and secondarily generated circumferential tensile force-, longitudinal stretching force- and compressive force. In particular, plaques in the coronary arteries are exposed to considerable cyclic longitudinal stretching force during the cardiac cycle (Gross and Friedman. J Biomech Eng. 31(5):479-84, 1998; Ding and Friedman. J Biomech Eng. 122(5):488-92, 2000). Upon distension of the arterial wall, the plaque cap is exposed to large tensile force. It is also well established that cap shoulder rupture is the most common cause of atherothrombosis leading to acute cardiovascular events. Thus, these biomechanical forces are considered extrinsic factors, triggering a potential rupture of the vulnerable plaque (Richardson. Ann Biomed Eng. 30(4):524-36, 2000; Arroyo and Lee. Cardiovascular Research. 41(2):369-375, 1999).

A stable plaque resistant to the extrinsic triggering forces is considered a beneficial characteristic of an atherosclerotic lesion. Thus, plaque stabilization treatment could be a potential therapeutic strategy, which in combination with established treatment-arsenal, may reduce and stabilize the clinical manifestation of acute cerebro- and cardiovascular events. Numerous association studies have been carried out to identify characteristics of vulnerable plaques using histological and immunohistochemical approaches. Several researchers have used “plaque stability index” as a quantitative approach to assess the plaque vulnerability (Ni et. Al., Cardiovascular Research. 41(2):376-84, 1999). This index is calculated through the ratio between macrophages, lipid core and smooth muscle cells, as well as elastin. For example, Jackson et al., (Atherosclerosis 154(2):399-406, 2001), measured stability of the brachiocephalic artery plaque in ApoE−/− mice by doing systematic histological analysis. They detected differences in frequency of silent ruptures and cap thickness in diet-manipulated ApoE−/− mice compared to control. However, most of these studies are time-consuming and of retrospective nature.

Rekhter el al., (Circ Res. 83(7):705-713, 1998) were the first to introduce a functional prospective plaque rupture model in rabbit. By measuring the pressure needed to rupture a formed plaque over a balloon catheter, they could show that hypercholestoremia weakened the plaque through degradation of collagen in the cap. Unfortunately, this is an artificial model because it requires introduction of a foreign body (balloon), which may influence plaque formation progression. Moreover, the complicated surgical procedure used in this strategy may introduce large methodological variation and require large number of experimental animals. This method is also not applicable in a murine model of atherosclerosis.

Numerous genetically modified (“knock-out” and “transgenic”) atherosclerosis-prone mouse models are now available, which provide animal models with human-like atherosclerotic lesions (e.g. Reardon and Getz. Mouse models of atherosclerosis. Curr Opin Lipidol. 12(2):167-73, 2001). ApoE and LDL receptor double-knock out mice (DKO) are one of the mouse models, which develop advanced atherosclerotic plaques with human-like plaque composition. These animals offer potential as surrogate human-relevant atherosclerosis models. However, as mentioned above, morphology-based plaque rupture models are often time-consuming and labour-intensive. Further, due to the methodological variability within conventional histological techniques, it is hard to determine dose-response relationships in pharmacological intervention studies.

Most association studies carried out in humans or animals are of retrospective approach, in which outcome data are often insufficiently characterized. Mortality or morbidity causes do not necessarily depend on atherothrombosis secondarily to any plaque ruptures. Thus, there is a need in the art for a robust, reproducible, functional model to study plaque stability in appropriate atherosclerosis-prone animals, which can also allow testing of potential atherosclerosis therapeutic agents. Preferably, this model will be plaque size independent.

There is also a need for validated and robust biomarkers, which are mechanistically associated with in vivo plaque size and/or plaque stability to identify risk patient and follow treatment effect. The novel plaque rupture assay taught herein will, optionally in combination with morphological studies and plasma marker analysis, allow one to validate and identify established and novel markers for plaque vulnerability in vivo.

Using a forced rupture strategy, the inventors have developed a novel biomechanical ex vivo plaque rupture model to test the over-all mechanical strength of an atherosclerotic plaque in a test animal such as the mouse. Further, this model allows simultaneous measurement of plaque size, rupture site, vessel stiffness in addition to rupture force. This model also allows one to test for therapeutic agents that can stabilise atherosclerotic plaques. Using, for example, ApoE and LDL receptor double-knock out (DKO) mice, which develop advanced atherosclerotic plaques with human-like plaque composition, the present model has also been used to study age-dependant changes in plaque size, vessel stiffness and plaque stability.

### DESCRIPTION OF THE INVENTION

According to a first aspect of the invention there is provided an ex vivo method for determining the force or pressure required to rupture an atherosclerotic plaque, comprising excising the plaque-burdened vessel from an animal, applying a force or pressure to the plaque or plaque-burdened vessel and measuring the amount of force or pressure required to cause plaque rupture.

In this aspect of the invention, the animal can be any animal including a human. In the case of a human it would of course be a cadaver.

In one embodiment, the force or pressure is applied to the abluminal side of the vessel on the other side of the plaque. In another embodiment a suction force may be applied, around and on top of the plaque (i.e. on the luminal side of the vessel).

In a further embodiment the vessel is an aortic vessel.

According to another aspect of the invention there is provided a method for determining the mechanical strength of a plaque comprising subjecting an excised plaque to pressure or force means and measuring the amount of pressure or force required to rupture the plaque. In a preferred embodiment the excised plaque is one that is within adjacent tissue, i.e. plaque containing tissue or vessel.

According to another aspect of the invention there is provided a method for determining the ability of a test compound to enhance plaque stability comprising administering a test compound to an animal possessing an atherosclerotic plaque or predisposed to develop atherosclerotic plaques for a suitable period of time, excising the plaque, subjecting the excised plaque to pressure or force means, measuring the amount of pressure or force required to rupture the plaque and based on this value relative to a control value assessing whether or not the compound enhances plaque stability.

According to another aspect of the invention there is provided a method for determining the ability of a test compound to enhance plaque stability comprising:
(i) administering a test compound to a non-human animal possessing atherosclerotic plaques;
(ii) excising a plaque containing vessel segment from the animal;
(iii) exerting a physical force to the plaque from step (ii);
(iv) measuring the amount of physical force required to rupture the plaque;
(v) repeating or carrying out steps (i) to (iv) but with a control animal that has not been treated with the test compound;
(vi) comparing the amount of plaque rupture force from step (iv) and (v) and, if the plaque rupture force from step (iv) is greater that that from step (v) identifying the test compound as one that enhances plaque stability.

This method can be used to assess whether or not a compound has potential in treating or preventing cerebro- or cardiovascular events due to plaque rupture such as primary or secondary myocardial infarction.

Physical force can be either tensile force (F) or hydrostatic pressure (P). Tensile force is expressed in Newton and hydrostatic pressure is expressed as mmHg. Force divided by surface area (A) is hydrostatic pressure. F/A=P.

In a preferred embodiment, the method involves application of a tensile force, such as for example by using a rod or piston. This method is an extremely sensitive method for measuring plaque rupture and is size-independent.

In a similar way as the force-method, the plaque-burdened vessel can be stretched in many different ways by exerting a pressure from a solid, liquid or gas against the inner vessel wall opposite the plaque site. Alternatively, a suction force can be applied directly to the plaque.

Thus, in an alternative embodiment, pressure means are used in the methods of the invention. According to one example of the pressure induced plaque rupture method, the set-up is such that a syringe is connected to the plaque holder. By pumping physiological salt solution (PSS) or other suitable liquid or gas at a constant speed, the vessel sheet is distended due to the increased pressure on the abluminal side of the vessel. The pressure developed during the infusion of PSS can be recorded using a pressure transducer (TPTT, Triplus, Sweden). The digital data acquisition system used in the current set-up is similar to the force-method.

Thus, in one embodiment, the pressure applied to the plaque in step (iii) is positive pressure effected by pushing a liquid (including gas) against the inner wall of the vessel opposed to the plaque. In another embodiment, the pressure applied to the plaque in step (iii) is negative pressure effected by sucking a liquid (including gas) that surrounds the outer wall of the vessel around the plaque.

In a further embodiment, the force applied is tensile force. Such force can be applied by contacting the surface of a solid member, such as a rod or piston made of any suitable material such as metal or plastic, to the innerwall of the vessel opposite the site of the plaque. This can either be accomplished by moving the solid support relative to a stationary, for example mounted, plaque containing vessel wall, or the vessel wall can be moved relative to a stationary solid support, or a mixture of both Such a setup is shown in Example 1, wherein by applying mechanical force on the abluminal side of the vessel wall, uniform tensile force is developed around the lesion. In vivo, an atherosclerotic lesion may be exposed to various magnitude of tensile force around the lesion shoulder. The set up disclosed in Example 1 does not discriminate different sites of the lesion and exposes the whole plaque shoulder to similar force. The first rupture site identified using this method, should thus be the most vulnerable spot of the cap shoulder. This strategy is particularly useful in pharmacological studies, e.g. plaque stabilization treatment, when the drug is supposed to exert overall stabilizing effect on the atherosclerotic lesions.

When using a force-method, the solid member is preferably a rod or piston, optionally of approximate cylindrical shape. In one embodiment, the force-exerting surface of the solid member is shaped so as to allow an approximately even application of force across the touching surface. A suitable surface shape is dome shaped. In another embodiment, the force-exerting surface of the solid support has approximately the same or larger diameter as the plaque on the opposed side of the vessel wall. In a preferred embodiment the diameter of the solid surface contacting the vessel wall is larger than that of the plaque on the opposed side of the vessel wall, such that the solid surface will exert a force by pressing against the plaque-burdened vessel as well as vessel tissue surrounding the plaque.

Thus, according to one particular embodiment, the force applied to the plaque is carried out by mounting the plaque-containing vessel in a holder and either (i) pushing a solid member against the inside of the vessel opposed to the plaque, or (ii) pushing the innerwall of the vessel opposed to the plaque against a solid member, and measuring the amount of force required to cause plaque rupture. In the set up of Example 1, the plaque holder is moved relative to a stationary rod. This allows the microscope that is situated above the plaque holder to remain focused on the rod surface. In a further embodiment, force is applied to the plaque by distending a plaque burdened vessel sheet over a force transducer which can then measure the amount of force applied to the vessel/plaque.

In one particular embodiment of the present invention as shown in the FIGS. 8 to 16 a compact device (12) is constructed from metal such as stainless steel. The components of the device are preferably constructed using suitable metal rods which are drilled and machined as required. Less preferably they may be cast and then machined. Those skilled in the art to which the invention relates will realise that other suitable alternative materials may exist for example, hard machineable plastic. The form of the device for use in the invention as illustrated in FIGS. 8-16, essentially comprises three units. A base holder (10), a cap (11) and a solid penetrating rod or piston to provide a rigid member (not shown) that moves co-axially within the interconnected holder and cap so as to push against the tissue sandwiched between the holder and cap, causing rupture of the plaque. The tissue can be placed over the narrow end of the base holder unit (13). In this form of the invention the rigid member is driven forward by means of a micro motor and a force displacement transducer provides a measurement of the force required to rupture the material. A number of suitable components can be used to provide the micro motor and the force displacement transducer, it is useful that they are both compact in size, able to cope with the environment and have the ability to provide consistent results. In preferred forms of the invention the various components are sealed by means of silicone sealant. O-rings of a suitable material may provide a useful alternative. In either case the sealing material may provide a degree of dampening of the device in use, which may allow more accurate results to be obtained. The rod or piston is one of a range of sizes but it has been found that a rod or piston having a diameter of approximately 30 to 98% of the diameter of the core unit of the rupture device provides good results. The diameter of the device can, again, be selected from a range and it has been found that a diameter of from 1 to 3 mm provides useful results but that the diameter may be at little as 0.5 mm or as much as 19 mm. The surface of the rupture rod or piston which contacts the material to be tested should at least have rounded edges or preferably be curved so that sharp edges do not damage the surface of the material to be tested prior to rupture. It has been found that the curvature of the piston can usefully be 10 to 80% of the device diameter. The form of the invention as shown in FIGS. 8 to 16 provides a number of projections (20, 21, 22, 23) in this case 4, which allow the holder and cap units (and sandwiched tissue) to be secured by means of elastic or rubber bands or other flexible materials. The holder and cap may be connected by an interference fit, however, it will be appreciated that other ways of holding the two units together, including screw/thread means, would work.

Plaque rupture can be detected visually, for example with the aid of a suitable microscope. Alternatively, plaque rupture can be visualised on a monitor connected to a camera directed at the plaque. Alternatively, plaque rupture can be determined by deviation in first derivative of the force development. First derivative (dF/dt, F=force; t=time) of the force developed upon distension of a plaque burdened vessel wall is typically exponentially incremental before the plaque ruptures. Once the plaque cap ruptures, the first derivative typically shows a slower increase. This change in slope of the first derivative indicates the plaque rupture event, which can also be validated by visual inspection.

The test animal can be any non-human animal, including for example, mouse, rat, gerbil, hamster, rabbit, dog, cat, monkey, pig, sheep, horse, cow and goat that possesses atherosclerotic plaques. Because of their size and average life span small animals, such as rodents, are particularly useful. One of the preferred test animals is the mouse.

Such animals or strains of animals may exist naturally. Atherosclerotic plaques can also be induced in the animal by dietary methods known to the person skilled in the art. Alternatively, atherosclerotic plaque containing animals can be generated using knockout technology to inactivate specific genes whose inactivation lead to atherosclerotic plaque formation. A large number of single, double and further multiple knockout mice are available which develop human-like atherosclerotic plaques (see for example, Breslow Science. 272:685-688, 1996; Plump et al. Cell. 71:343-353, 1992; Reardon and Getz. Curr Opin Lipidol. 12(2):167-73, 2001). Knockout animals, particularly those wherein either or both of ApoE and LDL receptor have been knocked-out are of particular utility. Double knockout (DKO) animals wherein both ApoE and LDL receptor have been knocked out are suitable examples.

The generation of atherosclerotic plaque susceptible transgenic or knockout animals is not part of this invention. However, such animals can be created using standard procedures as outlined briefly below and as described for example in “Gene Targeting; A Practical Approach”, IRL Press 1993. The target gene (e.g. Apo E, E4, E3, E2, LDLr etc.) or portion of it is preferably cloned into a vector with a selection marker (such as Neo) inserted into the gene to disrupt its function. The vector is linearised then transformed (usually by electroporation) into embryonic stem (ES) cells (e.g. derived from a 129/Ola strain of mouse) and thereafter, homologous recombination events take place in a proportion of the stem cells. The stem cells containing the gene disruption are expanded and injected into a blastocyst (such as for example from a C57BL/6J mouse) and implanted into a foster mother for development. Chimaeric offspring can be identified by coat colour markers. Chimeras are bred to ascertain the contribution of the ES cells to the germ line by mating to mice with genetic markers, which allow a distinction to be made between ES derived and host blastocyst derived gametes. Half of the ES cell derived gametes will carry the gene modification. Offspring are screened (e.g. by Southern blotting) to identify those with a gene disruption (about 50% of progeny). These selected offspring will be heterozygous and therefore can be bred with another heterozygote and homozygous offspring selected thereafter (about 25% of progeny). Transgenic animals with a gene knockout can be crossed with transgenic animals produced by known techniques such as microinjection of DNA into pronuclei, sphaeroplast fusion (Jakobovits et al. (1993) Nature. 362:255-258) or lipid mediated transfection (Lamb et al. (1993) Nature Genetics. 5:22-29) of ES cells to yield transgenic animals with an endogenous gene knockout and foreign gene replacement.

ES cells containing a targeted gene disruption can be further modified by transforming with the target gene sequence containing a specific alteration, which is preferably cloned into a vector and linearised prior to transformation. Following homologous recombination the altered gene is introduced into the genome. These embryonic stem cells can subsequently be used to create transgenics as described above.

The background strain of animal is a matter of choice. For example, with mice, the ApoE knockout trait has been bred into several different inbred mouse backgrounds, including C57BI/6, FVB/N, Balbc, C3H, C57BI/Ks, Cast/Ei and DBA/2J.

In one embodiment of the invention, the test animal is a single knockout (SKO) animal. In another embodiment the test animal is a double knockout (DKO) animal. In another embodiment the test animal is a mouse wherein both ApoE and LDL receptor have been knocked out. In a preferred embodiment the mice used are ApoE−/− and LDL receptor−/− mice. Such mice are commercially available from various sources such as M&B, Ejby, Denmark.

As disclosed in the Examples herein, the invention can be carried out using any plaque-containing (plaque-burdened) vessel. The inventors have found that with mice, despite considerable size variation, plaques sited on the 4th to 7th intercostal branches demonstrate the lowest inter-individual variation regarding the absolute rupture force. Therefore, in a particular embodiment, when applied to mice, the methods of the invention use plaques that are located on the 4th to 7th, inclusive, intercostal branches. In another embodiment the methods of the invention use plaques that are sited on the 6th intercostal branch.

Intercostal vessels branch from the dorsal side of the aorta pairwise and provide the intercostal rooms with blood-supply. Atherosclerotic plaques form typically over the branch sites at the dorsal side of the aorta. There are typically 11 pairs.

The terms “sited” and “located” are used interchangeably herein.

In one embodiment, only one plaque is tested per animal. In another embodiment more than one plaque, for example 2, 3, 4, 5 or more, is tested from each animal. In another embodiment, plaques from the same location(s) from numerous animals of the same strain and approximate age are tested. The design of such experiments, including the number of plaques, size of plaques, site of plaques etc. is a matter of choice for the person skilled in the art.

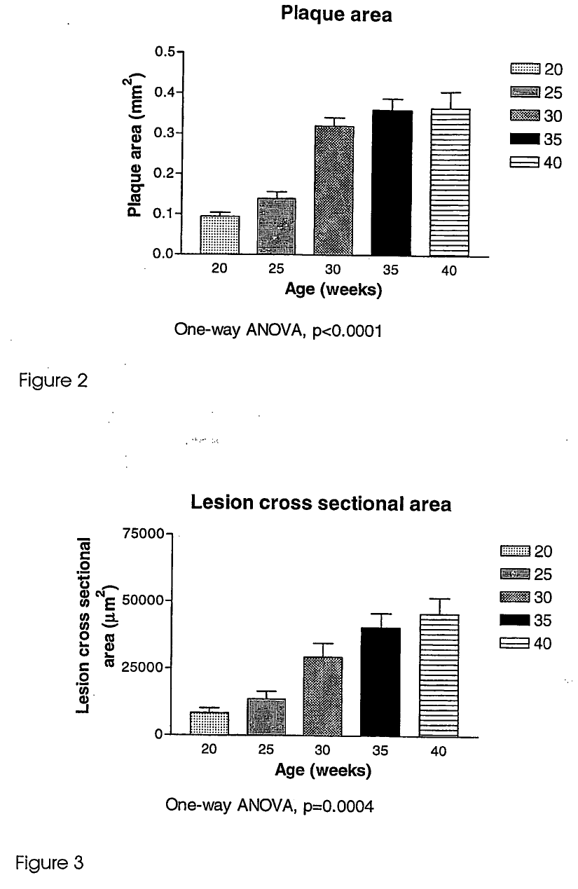

Provided atherosclerotic plaques are present, the actual age of the test animal is immaterial. However, the inventors have found that various characteristics of plaques and vessel walls housing these change with age. For example, overall average mechanical strength of plaques decreases with age (see FIG. 5). Plaque area increases with age, but then reaches a plateau (see FIG. 2). Lesion cross-sectional area also increases with age (see FIG. 3). Also, the vascular stiffness of the non-plaque burdened vessel wall increases significantly with age, in mice reaching a plateau at about 35 weeks. Plaque size also stabilises with age. With this knowledge, and depending on what application the methods of the invention are to be put to, the operator can select the age of the animal that exhibits the most appropriate characteristics.

Accordingly, in one embodiment of the invention the test animal is of an age when the average plaque size has stabilised. The particular age when plaque size stabilises in a species or particular strain of animal can be determined according to the examples described herein adapted to whatever species or strain of animal is under study. In another embodiment, it may be desirable to perform the methods of the invention on plaques that are still growing in size. In such instances, younger animals would be used.

In one embodiment, the test animal is a mouse younger than 35 weeks old. In another embodiment the test animal is a mouse is at least 20 weeks old, preferably at least 25 weeks old, more preferably at least 30 weeks old and still more preferably at least 35 weeks old.

The inventors have determined that the methods of the invention that measure tensile force are plaque size independent. Nevertheless, when using the methods of the invention, the person skilled in the art may elect to use/study plaques of approximately the same size. For example with mice, suitable plaques may possess a plaque area between 0.1 and 0.5 mm², preferably between 0.2 and 0.4 mm².

When testing the effect of a compound on plaque stability it is preferred, though not essential, that replicate experiments are performed. Usually, at least 10 plaque rupture experiments will be conducted per test compound to establish a mean rupture force that is of statistical value.

According to a further aspect of the invention there is provided for the use of an excised plaque or plaque-burdened vessel in an assay to measure plaque rupture or plaque stability.

According to a further aspect of the invention there is provided the use of an atherosclerotic plaque lesion possessing animal in an ex vivo plaque rupture assay.

According to a further aspect of the invention there is provided a method for determining whether or not a compound has potential in treating or preventing cerebro- or cardiovascular events due to plaque rupture, comprising testing the ability of the compound to enhance plaque stability in an ex vivo plaque rupture assay. Examples of cardiovascular events due to plaque rupture are primary or secondary myocardial infarction. Examples of cerebro- events due to plaque rupture are stroke and transient ischemic attack (TIA).

According to a further aspect of the invention there is provided a method or device substantially as described in the description, drawings (figures) or examples.

According to another aspect of the invention there is provided a device substantially as hereinbefore described with particular reference to one or more of FIGS. 8-16.

The invention will now be described by way of the following non-limiting examples and figures, wherein:

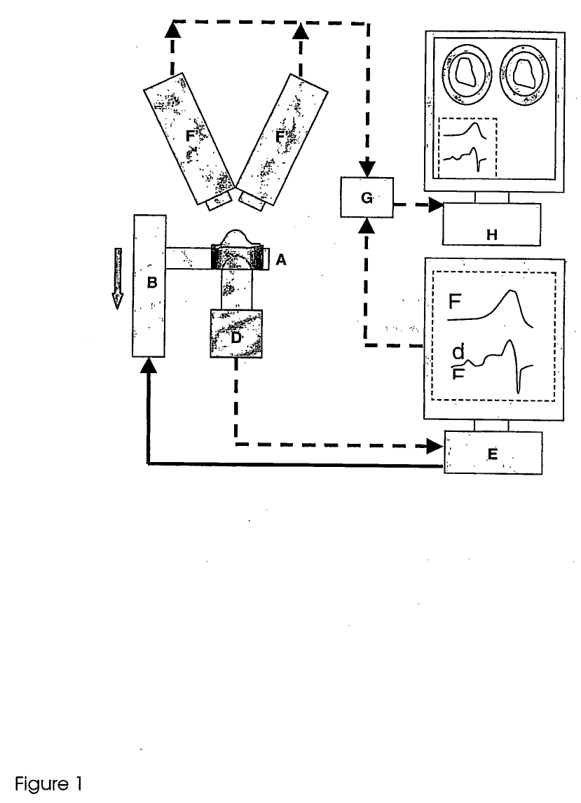

FIG. 1 represents a schematic of the experimental set up used in Example 1. The plaque burdened vessel sheet is mounted on the plaque holder (A), which is connected to the micromotor (B). Force is applied on the abluminal side of the vessel sheet through the piston (C), which is connected to a micro force transducer (D). Force developed during the rupture run is recorded in a personal computer (E), which also controls the movement of the micromotor. The rupture run is imaged in real-time through two angled zoom lenses (F′). Both the image signals and the force values are synchronized through a 16-channel digital disk recorder (G) and presented in a separate display (H).

FIG. 2 shows that plaque size increases with age, reaching a plateau in the mice under study at about week 30.

FIG. 3 shows that lesion cross sectional area increases with age.

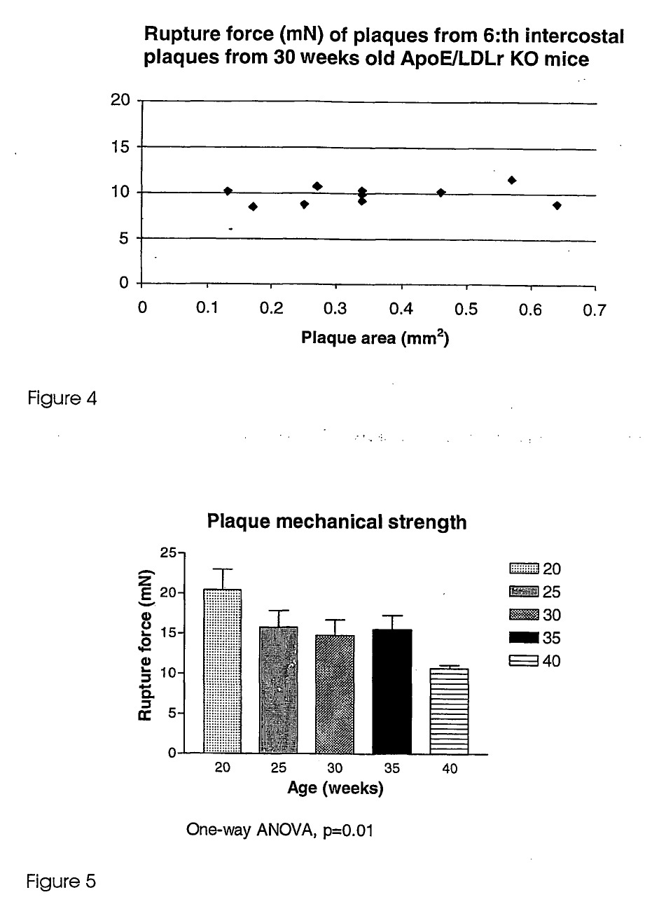

FIG. 4 shows similar rupture force of plaques from 6:th intercostal branch site from 30 weeks old DKO mice despite large variation in plaque size, which ranges from 0.12 to 0.64 mm² in plaque base area.

FIG. 5 shows average rupture force values for the five age groups of mice.

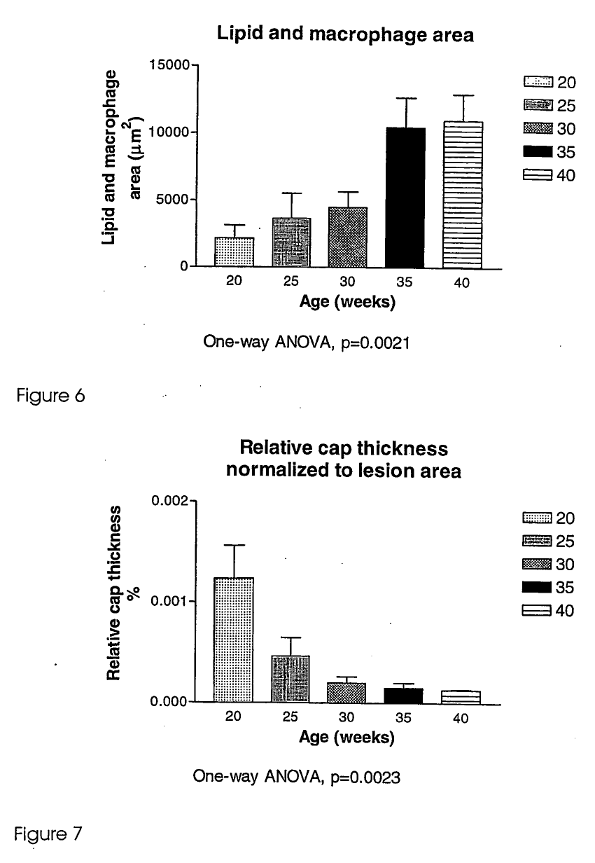

FIG. 6 shows that lipid and macrophage area increases significantly with age.

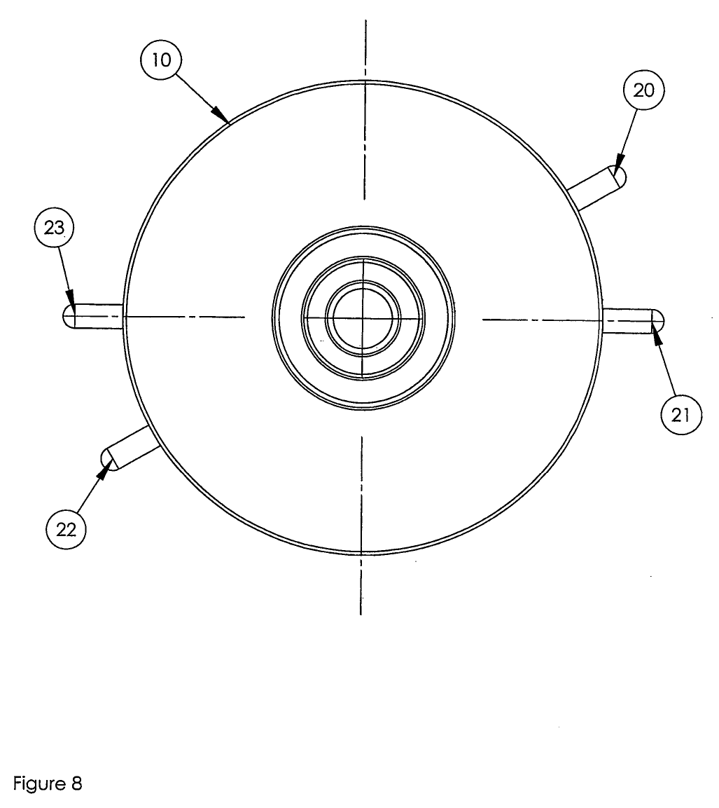

FIG. 7 shows that normalized vessel stiffness increases significantly with age.

FIG. 8 shows a plan view of a plaque holder base unit according to at least one form of the present invention.

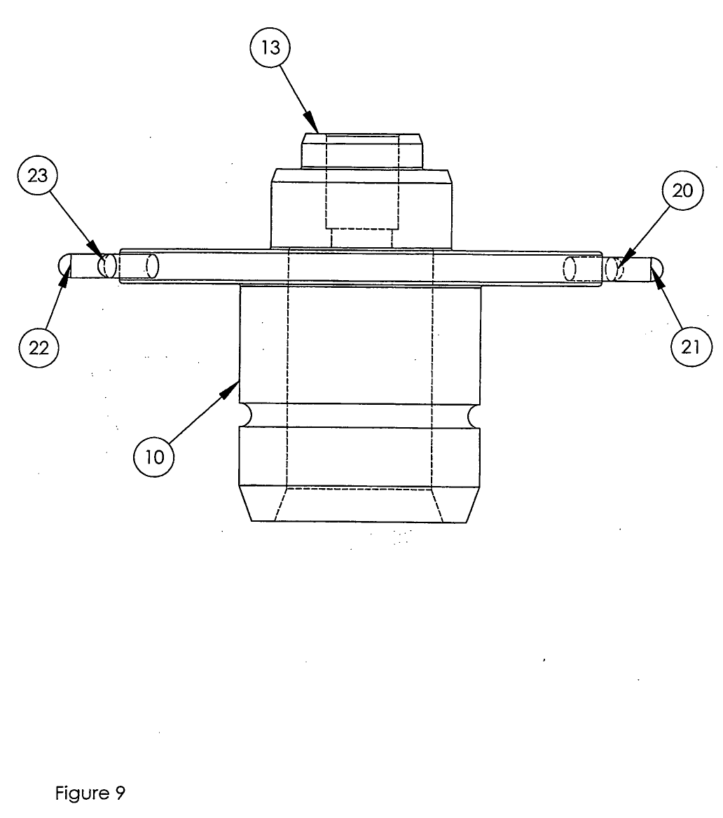

FIG. 9 shows a side view of a plaque holder base unit according to at least one form of the present invention.

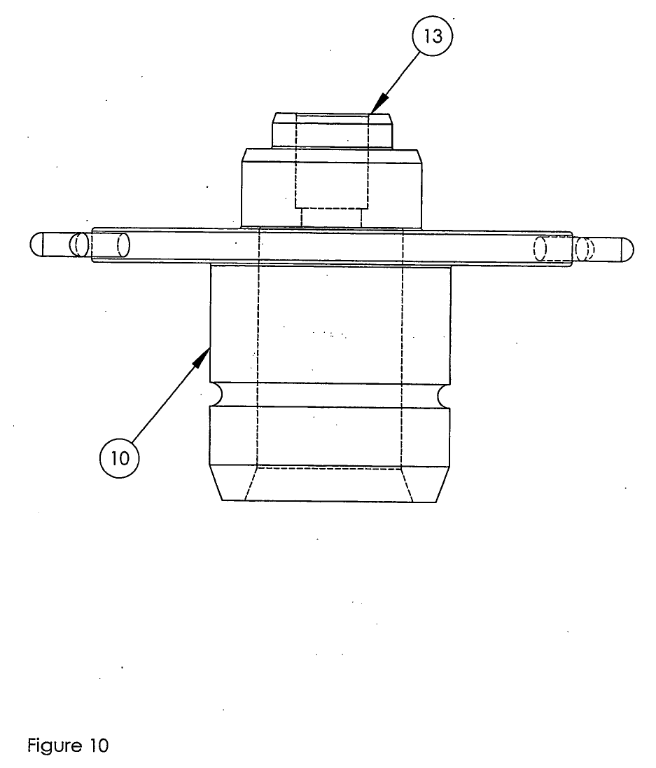

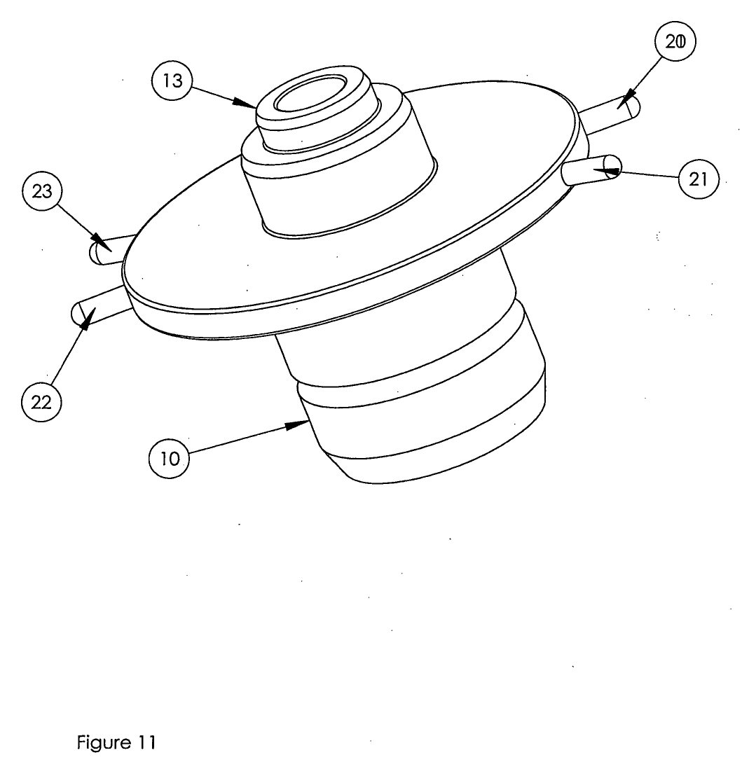

FIG. 10 shows a further side view of a plaque holder base unit according to at least one form of the present invention.

FIG. 11 shows a perspective view of a plaque holder base unit according to at least one form of the present invention.

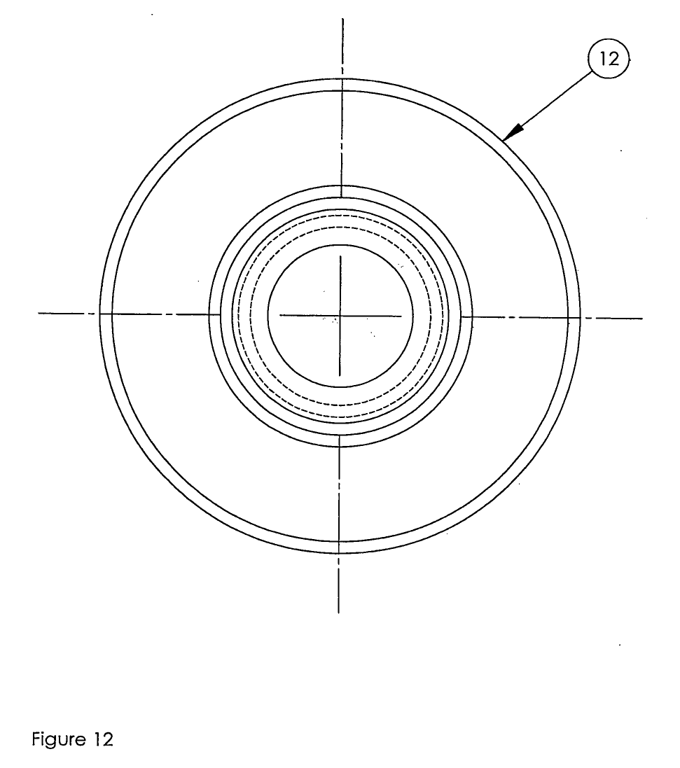

FIG. 12 shows a plan view of a plaque holder top (cap) unit according to at least one form of the present invention.

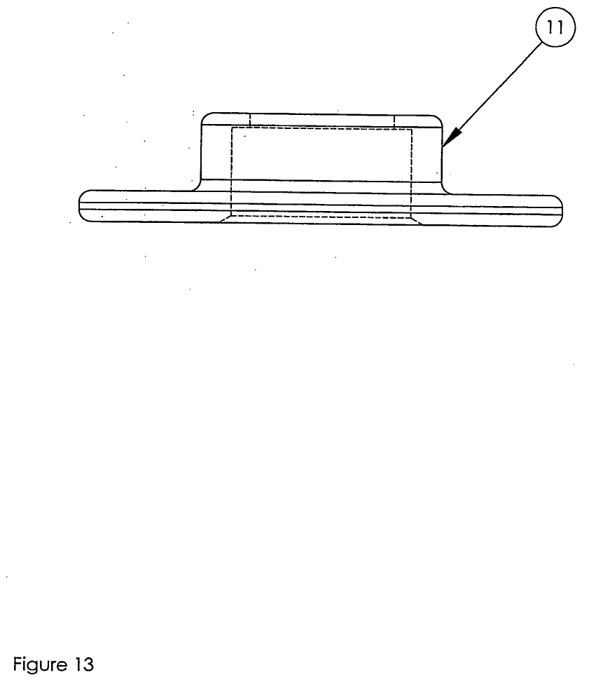

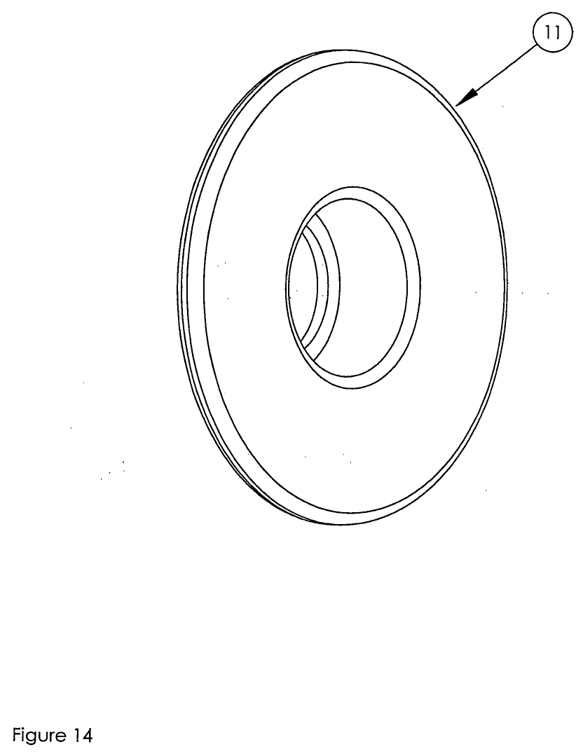

FIG. 13 shows a side view of a plaque holder top unit according to at least one form of the present invention.

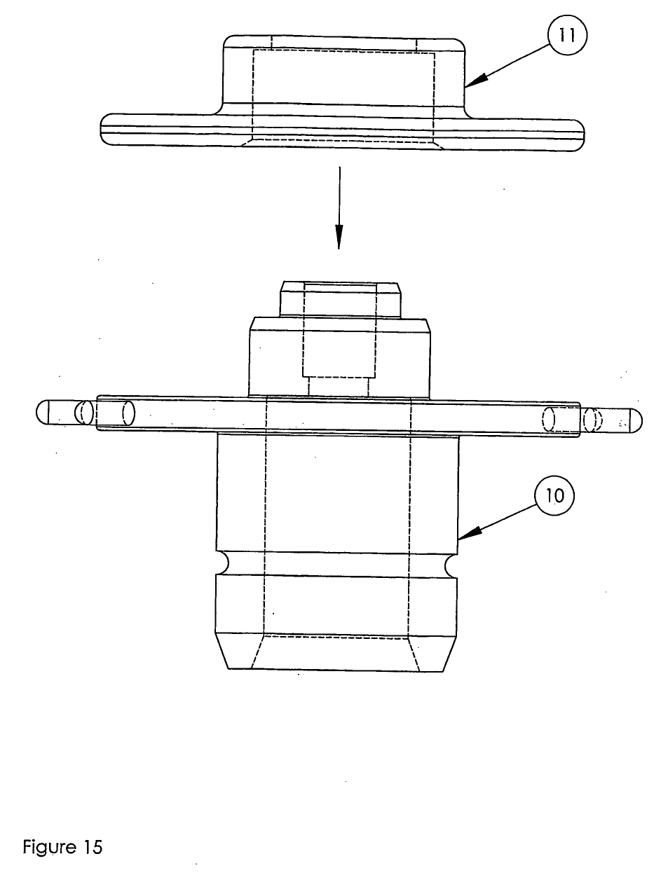

FIG. 14 shows a perspective view of a plaque holder top unit according to at least one form of the present invention.

FIG. 15 shows a side view of a plaque holder base and top unit according to at least one form of the present invention, with arrow indicating how they would fit together.

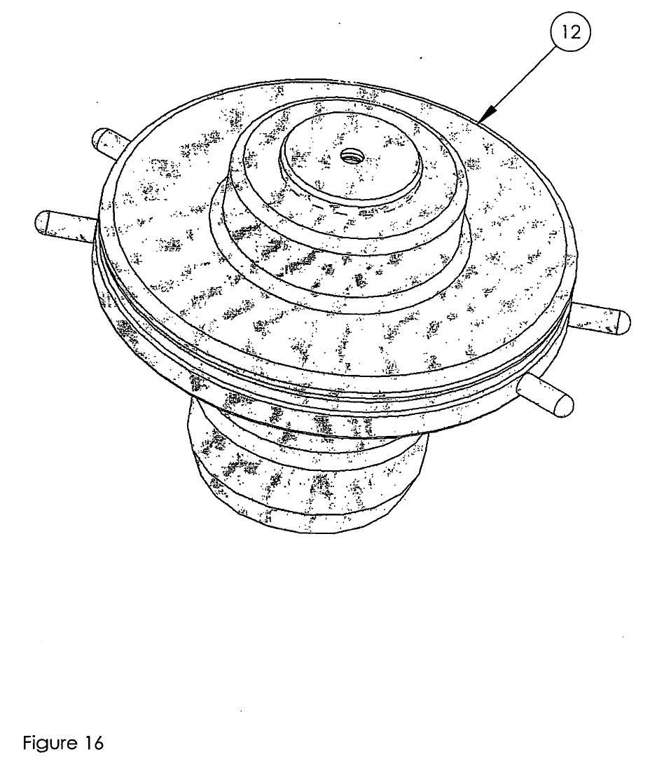

FIG. 16 shows a perspective view of a plaque holder base and top unit fitted together according to at least one form of the present invention.

### MATERIAL AND METHODS

Animals

Five different aged groups (20, 25 30, 35 and 40 weeks) of ApoE−/− and LDLr−/− double-knock out mice were purchased from M&B (Ejby, Denmark). An additional group of seven C57/B16 mice were used as normal control (M&B, Ejby, Denmark). Animals were acclimated for at least one week after arrival to the laboratory before onset of the experiment. All animals were housed at constant temperature (24° C.) at a relative humidity of 50-60%. A 12-hour dark/light cycle was maintained in the animal room. The mice had free access to standard pellet chow and tap water. Animals were weighed weekly. The experimental protocol was approved by the Regional Animal Ethic Committee, Göteborg University.

Experimental Protocol

The mice were anesthesized with Isofluran gas and killed by opening of the thoracic cavity. Blood sample was taken from the left ventricle immediately before the cardiac arrest and the circulatory system was thereafter rinsed with physiological salt solution (PSS). The thoracic aorta was carefully dissected free off from adjacent tissue and cleaned. Thoracic aortic plaques were typically present at intercostal artery branch sites. In the initial variability study, plaques from all branch sites were used. In the subsequent comparative study, only intercostal plaques between 4th and 7th branch sites (inclusive) were selected. A 3 mm vascular segment containing the plaque to be studied was excised and cut along the ventral side of the vessel, to create a vessel sheet with an isolated plaque in the middle. This approach facilitates imaging of the branch site plaque during the experiment. A droplet of PSS was applied on the plaque to protect the vessel from dryness before the vessel sheet was mounted on the plaque holder.

Description of Plaque Holder

The plaque holder used in this example is illustrated in FIGS. 8-16. FIGS. 8-15 are in 10:1 scale. In practise, the plaque holder is two units comprising a base and a cap (top), which interconnect to sandwich the tissue in between. Both components are approximately cylindrical in shape. In use a rod or piston moves co-axially within the interconnected base and top unit, starting at the base and moving towards the top unit, to push against (exert a force) the sandwiched tissue.

Experimental Setup

After mounting of the vessel sheet on the plaque holder, the luminal side of the vessel was exposed for video imaging in real-time, using two angled zoom lenses (Hirox MX-5030SZII, 60×-300× Straight-view Lens, Hirox CO. Ltd., Japan) connected to their respective digital camera units (Hi-Scope KH-1000, Hirox CO., Ltd., Japan). Any suitable camera equipment capable of imaging murine plaques with sufficient resolution could have been used. The digital image signals from the main camera units were thereafter acquired, using a personal computer equipped with frame grabber hardware and an image analysis software package (Matrox inspector 3.1, Matrox Electronic Systems Ltd., Canada). This digitizing equipment and software can be substituted with any other kind of image acquisition hardware and image analysis software, e.g. image hard- and software from National instruments.

The abluminal side of the vessel was in touch with a piston connected to a micro force transducer (Force Displacement Transducer FT03, Grass, USA). Vertical position of the plaque holder can be accurately controlled by a micromotor (M-227,25, Physik Institute (PI), Germany) through a personal computer equipped with a control software (PI Mercury Controller 1.20, EC Hannover GmbH, Germany). This setup enables the plaque holder to move continuously at a predefined constant speed toward the fixed piston during the rupture run. The force developed when the vessel sheet is distended over the piston was measured by the force transducer and digitized through a personal computer equipped with a data acquisition hardware (National instruments Ltd, Austin, USA) and a software package developed by AstraZeneca (Pharmlab, AstraZeneca R&D, Sweden). This software also provides on-line calculation of the first and second derivatives of the acquired force value during the experiment run to detect possible discontinuity in force development. This strategy can be carried out by a number of alternative data acquisition hardware and software, which can digitize and collect an analog signal and perform first derivative calculation on-line of the collected signal (e.g. from National Instruments, Austin).

To be able to correlate visible plaque rupture events with changes in force derivative, all video images and the data acquisition interface were synchronized by being simultaneously shown in a separate display with split-screen mode and stored in a 16-channel digital disk recorder (WJ-HD500A, Panasonic Ltd., Japan). Although this is an optional feature, this setup facilitates off-line investigation of rupture events and force development with high time-resolution.

The set up used in this Example is illustrated in FIG. 1.

Measurement of Vessel Stiffness at the Plaque Site

All rupture run video sequences revealed that the non-plaque burdened vessel wall is stretched first during the rupture run. To test the wall stiffness of the non-plaque burdened vessel wall, we measured specifically the initial and final (plaque rupture moment) micromotor position and force developed at both positions normalized to the total area of the non-plaque burdened vessel wall. The following formula for vessel stiffness was used: (F₂-F₁)/(D₂-D₁)/(vessel area without plaque), where F₁ and F₂ are the force value expressed in mN before and at the end of the rupture run, respectively, and, D₁ and D₂ are micromotor position (mm) at the beginning and end of the rupture run, respectively.

Typically, the vessel sheet is stretched 0.5 mm in 30 sec or the assay is interrupted before the force reaches 100 mN.

Histology/Immunohistochemistry

After the rupture run, the visually detected rupture site was marked with tissue marking dye (black H-TMD-BK, TBS, USA), and the vessel tissue was immediately fixated in phosphor-buffered formaldehyde (HistoLab Products AB, Sweden). The fixated plaques were then embedded in paraffin and serial sections were performed over the marked rupture sites. Miller's staining (Histolab Products AB, Sweden) was used to visualize the elastic components of the plaque and cap rupture. Picrosirius red (Histolab Products AB, Sweden) was used to detect plaque content of collagen and smooth muscle cells. Fibromuscular cap thickness was calculated using this staining as collagen and smooth muscle cells are stained with red and yellow, respectively. By using a polarization filter, collagen type I and III can be differentiated (Junqueira et al., Archivum Histologicum Japonicum—Nippon Soshikigaku Kiroku. 41(3):267-74,1978). The unstained area was regarded as lipid and macrophage area. Anti-mouse Mac-2 monoclonal antibody (Clone M3/38, Cedarlane, Canada) was used for staining of macrophages.

Computerized Morphometry

All sections were examined on a light microscope and images were digitized and measured in ImagePro analysis software (Image Pro Plus 4.5, Media Cybetietrics, USA). Cap thickness was measured in a blinded manner at three different localizations of the plaque cross sectional images. The cross sectional area and maximum height of the plaques were measured. Collagen content in red polarization filter, macrophage and lipid core area were measured by an automatic spectral recognition routine in the software according to a method described previously (Gan et al. Cardiovascular Research. 48(1):168-77, 2001). Briefly, the areas of positive staining were analyzed by a spectral analysis using the software and confirmed by visual inspection. Thereafter, the spectral values were stored and used for further detection of positively stained area.

Statistics

Kruskal-Wallis non-parametric one-way ANOVA was used to test the pattern of changes and Post-hoc analysis was performed only when the over-all p-value was significant using Dunn's multiple comparison test. P-value <0.05 was considered a significant change.

Druges

All chemicals were purchased from Sigma unless otherwise stated.

### RESULTS

Methodological Validation

Discontinuity in first rupture force derivatives was well correlated with visually detected plaque rupture event. Further, subsequent histological analysis revealed ruptured cap on intact media, which confirms that this experimental set-up caused the plaque cap to rupture. Force and force derivative development patterns were distinct for healthy compared to plaque-burdened vessels, as the former compared to the later typically did not show any discontinuity in the first force derivative.

Although the assay method can be performed with plaques from any location, by studying rupture force of plaques from different aortic locations, the inventors found that plaques from 4th to 7th intercostal branch sites showed lowest inter-individual variation with regard to the absolute rupture force, despite considerable size variations (data not shown).

Size Independent Method

Mechanical strength from intercostal plaques from the 6th branch site were studied from ten 40-week old ApoE and LDL receptor double-knock out (DKO) mice. Despite the relatively large size variation, rupture force was found to be similar in all these plaques. A correlation was found between plaque size and rupture force, which confirms that the method is a size independent technique (FIG. 4).

Change of Plaque Size by Age

The plaque area measured by video-imaging was found to increase significantly with age (p<0.0001) (FIG. 2). Lesion-cross sectional area assessed by histology and subsequent morphometric analysis showed significant age-dependant increase (p=0.004) (FIG. 3).

Age Dependant Change of Rupture Force

Overall mechanical strength was found to decrease significantly with age (p=0.01). Average rupture force was 20.4±2.7 mN in 20-week old mice and levelled off at 15.8±2.1 mN in 25-week and older mice. This value remained unchanged until week 40, when the average rupture force further decreased significantly to 10.7±0.4 mN (p<0.05, w40 vs w35). See FIG. 5.

Vessel Stiffness Change by Age

The vascular stiffness of the non-plaque burdened vessel wall increases significantly with age (p=0.0043) reaching a plateau after week 35.

Compositional Data

Plaques from the 6th intercostal region showed characteristics, including a fibromuscular cap, of a typical human atherosclerotic lesion. Cap shoulder region is typically rich in macrophages and lipid pool and poor in collagen under polarization filter. These characteristics are more pronounced in plaques from elder mice (FIG. 6).

In 20-week old DKO mice, the fibromuscular cap identified by Picro-sirus red staining was significantly thicker than the caps in older mice, and the cap thickness decreased significantly with age (p=0.0023) (FIG. 7).

Unstained area when stained with Picro-sirus red, identified the lipid and macrophage containing region in the plaques. Lipid and macrophage content in the studied plaques increases significantly by age (p=0.0021) (FIG. 6).

### DISCUSSION

The inventors have developed a novel ex vivo biomechanical model to test plaque mechanical strength. The model turned out to be a rapid, reproducible and size-independent method to evaluate the intrinsic properties determining plaque stability. Histological analysis verified cap shoulder rupture as the rupture mechanism in the model. Aortic plaques showed age dependant increase in both lesion base area and plaque cross sectional area, which suggests an increase in plaque volume with age. Plaque mechanical stability is decreased with age, while the non-plaque burdened vessel wall is getting stiffer with time. Histological analysis of the studied plaques revealed more accumulation of lipids and macrophages with age, preferentially at the lesion shoulder regions, meanwhile average cap thickness was decreasing. These compositional changes could be the plaque intrinsic properties responsible for a more unstable phenotype by age.

With the current exemplary set up, plaque rupture events were identified both visually and through slope changes in force derivative. This observation/invention makes the rupture identification more reliable and accurate. Since the elastic modulus in the plaque-free vessel wall and the plaque is distinct, the earliest time point when the force derivative changes its slope indicates the very first rupture event. In vivo, the very first rupture event is supposed to be a crucial initiating factor in an atherothrombotic event, since the flowing blood may further invade the fissured plaque and cause intra-plaque hemorrage, which may in turn accelerate the acute plaque rupture process and the subsequent acute thrombotic event (Robbie and Libby. Ann NY Acad Sci. 947:167-79, 2001; Richardson. Ann Biomed Eng. 30(4):524-36, 2000; Arroyo and Lee. Cardiovascular Research. 41(2):369-375, 1994).

Another interesting feature of the model is that stiffness of the studied vessels can be measured without any additional experimental steps. This was made possible by using a combination of high-precision force transducer and mechanical devices. The inventors found increased vessel stiffness of the non-plaque burdened vessel wall with age. This indicates that not only the plaque burdened vessel area is restricted in its distension during the cardiac cycle, but also the adjacent vessel tissue is getting stiffer, showing that progression of atherosclerosis is associated with decreased vessel compliance.

Compared to the Rabbit-balloon model disclosed by Rekhter (ibid), which is an elegant method for testing of rabbit plaque stability, the inventors have now created a novel plaque rupture model specialized for testing plaque mechanical properties, even though the model is exemplified using mice, it is also applicable to testing the mechanical strength of plaques from other species. This model also comprises several additional advantages. First, the normal lesion progression is not affected by using this ex vivo approach. Compared to plaques formed over an implanted foreign catheter, only naturally occurring plaques are studied in our model, which increases the biological relevance of this plaque rupture model. Second, tissue material from the experimental animals other than the studied plaques can be readily used for further research purposes, which improves the effectiveness of the utilization of experimental animals. Third, this method is rapid and uncomplicated, and does not require any pre-preparation of the animals, such as surgical modifications. All these advantages make the model highly relevant and useful in plaque stability research.

In summary, age-dependant destabilization of murine aortic plaques seems to be associated with accumulation of lipid and macrophages at the lesion shoulder regions and a relatively decreased fibromuscular cap thickness. The novel biomechanical ex vivo plaque rupture model is a useful tool for rapid and accurate determination of plaque mechanical strength.

### EXAMPLE 2

### Assessing Plague Stabilising Effect of Test Drugs

The plaque rupture assay described herein and in Example 1, can be used to assess the plaque stabilising effect of a test compound according to the following representative study design:

Four groups of 25 week-old DKO animals (12 in each group) are used. The test compound can either be administered orally in the chow or via lavage or via minipump, in three dose steps, to three of the groups and the fourth group serves as a control group. The study proceeds for a suitable period of time, for example 10 weeks. The animals are weighed weekly and plasma concentration of the compound is analysed, for example every second week.

At the end of the study, the animals are killed and the thoracic aorta is excised and the 6th intercostal plaque is dissected free and cut along the ventral side of the vessel to create a vessel sheet with the studied plaque in the middle. The vessel sheet is then mounted on the plaque holder (as described in Example 1). Total plaque area is measured. Thereafter the plaque rupture assay, as described in Example 1, is performed. Rupture force is measured. By studying mean values of rupture force from each group, possible differences in mechanical strength and dose response relationship can be detected.

1. A method for determining the mechanical strength of an atherosclerotic plaque comprising subjecting an excised plaque to force or pressure means and measuring the amount of force or pressure required to rupture the plaque.

2. (canceled)

3. A method for determining the ability of a test compound to enhance plaque stability comprising:
administering a test compound to an animal possessing an atherosclerotic plaque,
excising the plaque,
subjecting the excised plaque to pressure or force means,
measuring the amount of pressure or force required to rupture the plaques, and,
based on the amount of pressure or force relative to a control amount, assessing whether or not the compound enhances plaque stability.

4. A method for determining the ability of a test compound to enhance plaque stability comprising:
(i) administering a test compound to a non-human test animal possessing atherosclerotic plaques;
(ii) excising a plaque containing vessel segment from the animal;
(iii) applying physical force to the plaque from step (ii);
(iv) measuring the amount of physical force required to rupture the plaque;
(v) carrying out steps (ii) to (iv) with a control animal possessing atherosclerotic plagues that has not been treated with the test compound;
(vi) comparing the amount of plaque rupture force from step (iv) and (v) and, if the plaque rupture force from step (iv) is greater that that from step (v) identifying the test compound as one that enhances plaque stability.

5. The method according to claim 4, wherein the test animal has been induced to develop atherosclerotic plaques by dietary feeding.

6. The method according to claim 4, wherein the test animal is a transgenic or knock-out animal.

7. The method according to claim 6, wherein the knock-out animal is a single or double knock-out animal.

8. The method according to claim 6 or 7, wherein the test animal has a defunct LDL receptor and/or a defunct APO E receptor.

9-12. (canceled)

13. The method according to any one of claims 3 or 4, wherein the animal is a mouse, rat, gerbil, hamster, guinea pig, rabbit, dog, cat, monkey, pig, sheep, cow, or goat.

14. The method according to claim 4, wherein the physical force applied to the plaque in step (iii) is tensile force.

15. The method according to claim 4, wherein the physical force applied to the plaque in step (iii) is carried out by mounting the plaque in a holder and allowing a solid member to push against the inside of the vessel opposed to the plaque and measuring the amount of force required to rupture the plaque.

16. The method according to any one of claims 1, 3, or 4, wherein the amount of force is measured using a force transducer.

17-22. (canceled)

23. The method according to any one of claims 1, 3, or 4, wherein the plaque is an aortic plaque.

24. (canceled)

25. A method for determining whether or not a compound has potential in treating or preventing cerebro- or cardiovascular events due to plaque rupture, comprising testing the ability of the compound to enhance plaque stability in an ex vivo plaque stability assay.

26. The method according to claim 25, wherein the ex vivo plaque stability assay comprises subjecting an excised plaque to force or pressure means and measuring the amount of force or pressure required to rupture the plaque.

27. The method according to claim 25 or 26, wherein the cardiovascular event is primary or secondary myocardial infarction.

28. A device for determining the mechanical strength of a plaque comprising means for securing a plaque-burdened vessel and means for exerting a physical force on the plaque burdened vessel.

29. The device according to claim 28, further comprising means for measuring the physical force.

30-49. (canceled)
